(19) United States
(12) Patent Application Publication
(10) Pub. No.: US 2024/0105309 A1
(43) Pub. Date: Mar. 28, 2024

(54) SYSTEM AND METHOD FOR TREATING AND TRACKING MENTAL HEALTH USING BIO-PSYCHO-SOCIAL (BPS) FORMULATION

(71) Applicant: Anvesh Roy, Mississauga (CA)

(72) Inventor: Anvesh Roy, Mississauga (CA)

(73) Assignee: Psychnosis Inc., Mississauga, ON (CA)

(21) Appl. No.: 17/951,226

(22) Filed: Sep. 23, 2022

(51) Int. Cl. G16H 20/70; G16B 20/00; G16H 10/20; G16H 10/60; G16H 40/67; G16H 50/20; G16H 80/00

(52) U.S. Cl. CPC G16H 20/70; G16B 20/00; G16H 10/20; G16H 10/60; G16H 40/67; G16H 50/20; G16H 80/00

(57) ABSTRACT

A BPS Formulation is routinely used by mental health care clinicians to understand their client or patient thoroughly and come up with a comprehensive BPS treatment plan which is the gold standard in mental health care. A system and method for treating and tracking mental health using Bio-Psycho-Social (BPS) Formulation are disclosed. The system is configured to: collect a patient's psychiatric history including data on symptoms and relevant psychiatric history using evidence based questionnaires; collate the data into biological, psychological, and social categories using a database; create a unique BPS profile of the patient; analyze a link between the symptoms and BPS factors and known associations in psychiatric literature to generate a BPS Formulation and from this a Biopsychosocial treatment plan, and allow a medical professional to facilitate the treatment of patient with mental health problems and also inform or educate a patient or client regarding their unique BPS factors leading to mental health issues. The system re-calculates BPS formulation over time by repeating relevant questions at regular intervals, thereby tracking the mental health of the patient and providing real-time feedback to the patient and their health care provider to further enhance their recovery and treatment.

### FIELD OF THE INVENTION

The present invention generally relates to health monitoring systems. More specifically, the present invention relates to a system and method for providing a clinical support tool configured to treat and track the mental health of an individual using bio-psycho-social (BPS) formulation.

### BACKGROUND

A health monitoring system is a secure platform that connects healthcare providers with patients. It is a platform to collect and analyze healthcare data of patients at regular intervals. This system continuously monitors patient health and provides necessary actions to be followed. This system is further facilitated by direct interaction with doctors, physicians, and medical consultants with patients to know and get feedback about their health conditions in real-time. This monitoring system will significantly improve the treatment process and provides a simple and cost-effective remote health monitoring system.

A remote health monitoring system or virtual health monitoring system is a common practice followed when patients do not have access to specialists and psychiatrists nearby. These systems greatly support patients with timely health monitoring and immediate solutions to their problems. Moreover, this system helps the patients to feel comfortable and helps them to manage their own health.

Few existing patent references attempted to address problems cited in the background as prior art over the presently disclosed subject matter are explained as follows:

A prior art AU2021105750 of Galambos Gary, entitled “System and Method for Virtual Mental Health System Infrastructure” discloses a system and method for providing virtual mental health system infrastructure adapted to educate, refer, and support patients to specialist-grade digital mental health services. The system further provides an eLearning service includes autonomous learning principles with an innovative true visual learning approach with visual analogies, role play with conversing characters and avatars enabling a minimization use of technical words and jargon.

Another prior art US 20120221251 A1 of Saul Rosenberg et al., entitled “Systems and methods for selecting, ordering, scheduling, administering, storing, interpreting and transmitting a plurality of psychological, neurobehavioral and neurobiological tests” discloses a system comprises a validation module and an analysis module. The validation module is configured to receive input data from an electronic device operated by a test-taker during a biopsychosocial assessment. The input data includes a plurality of input terms that describes a functioning of the test-taker. The validation module is configured to validate the plurality of input terms based on a plurality of pre-defined terms stored within the system. The analysis module is configured to generate a list of potential biopsychosocial-related issues associated with the test-taker based on the validated input terms, and to generate a recommendation of one or more tests to administer to the test-taker based on this list. The analysis module is further configured to transmit an output signal representing the recommendation to an electronic device such that the one or more recommended tests are presented at an electronic device.

Another prior art US 20210343407 A1 of Kenneth Neumann et al., entitled “Methods and systems for dynamic constitutional guidance using artificial intelligence” discloses a system for dynamic conditional guidance using artificial intelligence. The system includes a computing device, designed and configured to calculate a diagnostic output using a biological extraction related to a user, and a first machine-learning process, wherein the diagnostic output identifies a prognostic label and an ameliorative label; classify, using a physiological classifier and a first classification algorithm, the diagnostic output to a physiological state for the user; generate a vector output for the physiological state for the user, using a clustering algorithm; receive a user input generated in response to the diagnostic output; update the vector output using the user input; and identify a recommendation for the user, utilizing the updated vector output.

Though the existing prior art discloses various system and method for virtual health analysis and guidance to support patients none of them disclose a system and method that tracks the person's symptom over time and activates a solution/suggestion. Further, a system providing a Bio-Psycho-Social (BPS) summary with a BPS plan based on the findings of the BPS formulation is not disclosed. Furthermore, the analysis does not pay attention to the Biopsychosocial precipitating, predisposing, perpetuating and protective factors, in other words, the temporal relationship of these factors to the symptom complex or psychiatric diagnosis is not delineated. Therefore, the above arts are not true to the way Biopsychosocial formulation is done by mental health professionals in practice, and the system of the present invention would closely emulate that process as taught in mental health professional universities and is of maximum benefit to the patient in this form.

In light of all the above-mentioned drawbacks, there is a need for a system and method for providing mental health monitoring of a patient. Also, there is a need for a system that provides a Bio-Psycho-Social (BPS) summary with a BPS plan based on the findings of the BPS formulation, which can be continuously reformulated based on the Biopsychosocial factors present at that point of time in the patient's life.

### SUMMARY OF THE INVENTION

The present invention generally discloses a clinical support tool. Also, the present invention discloses a system and method for treating and tracking the mental health of an individual using a bio-psycho-social (BPS) formulation.

According to the present invention, the system is a computer-implemented system executed in a network environment for treating and tracking mental health of a user/patient/customer using Bio-Psycho-Social (BPS) Formulation. The system runs in the computer-implemented environment configured to provide an online/digital assistance for users suffering from mental health issues. In one embodiment, the system is a clinical support tool for family doctors, primary care physicians, nurse practitioners and psychotherapists before the patient is referred to a psychiatrist who are the only professionals trained to make a comprehensive Biopsychosocial formulation and plan. Thereby eliminating the need for a lengthy wait for a psychiatrist and implementing a Biopsychosocial plan. In one embodiment, the system is a digital platform designed to artificially replicate what a psychiatrist does when they are assessing a patient, in a user-friendly way.

In one embodiment, the network environment comprises one or more user devices. The user device has an interactive user interface or graphical user interface. Each user device is associated with a user. The user may be an end-user, individual, patient, or customer. The customers may be general doctors, nurse practitioner (nurses who can prescribe medications) and social workers, therapists, psychologist, crisis workers, and internet therapists. In one embodiment, the user device is installed with a digital platform (i.e., PSYCHNOSIS application). In one embodiment, PSYCHNOSIS is a type of e-mental health application. In one embodiment, the digital platform may be an application software or mobile application or web-based application or software application. The system further comprises a communication network and a psychiatric data management system. The user device is configured to deliver digital mental health service. In one embodiment, the user device is enabled to access the psychiatric data management system via the network. In one embodiment, the user device enables the user to access one or more services provided by the system. In one embodiment, the user device is at least any one of a smartphone, a mobile phone, a tablet, a laptop, a desktop, and/or other suitable hand-held electronic communication devices. In one embodiment, the user device comprises a storage medium in communication with the network to access the psychiatric data management system. In an embodiment, the network could be Wi-Fi, WiMAX, wireless local area network (WLAN), satellite networks, cellular networks, private networks, and the like.

In one embodiment, the psychiatric data management system comprises a computing device and one or more databases in communication with the computing device. In one embodiment, the computing device is a server. In one embodiment, the computing device could be a cloud server. In one embodiment, the server could be operated as a single computer. In some embodiments, the computer could be a touchscreen and/or non-touchscreen and adopted to run on any type of OS, such as iOS™, Windows™, Android™ Unix™, Linux™, and/or others. In one embodiment, the plurality of computers is in communication with each other, via networks. Such communication is established via any one of application software, a mobile application, a browser, an OS, and/or any combination thereof.

In one embodiment, the database is in communication with the computing device via the network. In one embodiment, the database is accessible by the computing device. In another embodiment, the database is integrated into the computing device or separate from it. In some embodiments, the database resides in a connected server or a cloud computing service. Regardless of location, the database comprises a memory to store and organize certain data for use by the computing device.

In one embodiment, the computing device comprises at least one processor and a non-transitory memory unit or computer-readable medium or memory unit coupled to the processor. The memory unit stores a set of instructions executable by the processor configured to analyze various clinical factors such as predispose, precipitate, perpetuate, and protect, and presumptive diagnosis. The memory unit could be RAM, ROM (including EPROM, EEPROM, PROM). In one embodiment, the user devices are configured to access the services provided by the computing device via the network. In one embodiment, the computing device is configured to provide communication between the users and medical professionals in the digital platform to provide suggestions to the users to improve their mental health.

In one embodiment, the computing device is configured to collect data that pertains to a patient's psychiatric history including data on the symptoms and medical, psychological, and social history in the least time-consuming way. The user interface is designed to emulate a human being or mental health professional asking such questions so as to have a pleasant and even therapeutic experience. Further, the system removes biases in questions.

In one embodiment, the computing device is further configured to collate the collected data into biological, psychological, and social categories using a database like structure. In one embodiment, the computing device is further configured to compute BPS Formulation using algorithmic and/or Artificial Intelligence (AI). BPS Formulation is the gold standard in psychiatric and psychological fields to holistically understand the person's current mental health issues such as to why this person is presenting with these problems at this point in time. This framework then is used to come up with a Bio-psycho-Social Summary that would accurately reflect to the user, for example, what the person is going through and what are the different factors that go into producing such a result. The central assertion is that there is value in understanding what is the issue affecting someone and the presentation of this to the user is hypothesized to be therapeutic and extremely helpful in and of itself. In one embodiment, the BPS Formulation includes Statistical Manual of Mental Disorders (DSM) and International Classification of Diseases (ICD) and/or diagnosis. In one embodiment, the BPS Formulation Summary is presented according to the end-user/consumer, i.e., whether it is presented to the patient or to a health care professional.

In one embodiment, the computing device is further configured to create a unique phenotype or in colloquial language a ‘fingerprint’ for an individual using the BPS factors found. This phenotypical representation is plotted in different dimensions to make a 2D or 3D picture representation. In one embodiment, the computing device is further configured to compare the fingerprint using the AI or computer software against a database with known biopsychosocial associations. These “stock” phenotypical representations of BPS associations with symptom complexes are commonly found in the Psychiatric literature that indicate types of patterns of BPS problems or associative chains. For example, Biological Predisposing factor of genetic or family history of depression is linked with depression symptom complex and alcohol is precipitating or perpetuating for this symptom complex. This will then help to come up with a BPS formulation that is representative of the persons' problems and have solutions as per this “matched” phenotype. In one embodiment, a causal chain for each linked factor between the symptom complex and BPS factor will use a rule-based database for performing pattern matching.

In one embodiment, the computing device is further configured to analyze a link between the symptoms that split into patients' symptom complex or diagnosis with the BPS, and their improvements. In one embodiment, the medical professional also adds their dictation and the data can be exported to a preferred format for storing it and this will save time in medical record generation for the professional improving patient care as now the professional will spend less time documenting and more time supporting and interacting with the patient.

In one embodiment, the system is further configured to track the persons' symptoms over time, and activates a solution/suggestion using the application software. The system further refines the unique fingerprint. Some factors of the unique fingerprint will not change over time and others will, either improving or getting worse. Each new problem will cause a spike in the fingerprint figure which can meet a threshold to activate a solution/suggestion by the application software. The solutions will be presented in the form of a Biopsychosocial treatment plan based on the BPS Formulation and Summary which is again the gold standard way to help someone with the psychiatric or psychological problem. The BPS plan will be configured based on the end user i.e., whether it is the patient or the health care provider. The figure is represented visually to give the user an idea of the patient's overall mental health. One can compare this to the “health indicator” on a video game and that to improve their health, the player must find a med kit which is the solution suggested by the App. Once this is implemented and the person is feeling better the fingerprint will change to show the progress to further help the patient or their health care provider with new suggestions needed to do to improve the patient's overall mental health. Then it is matched constantly again to the stock data to come up with new formulations and solutions. Also, there will be a stock fingerprint indicating normal mental health to compare against which is individual specific by analyzing what the realistic goals are for the particular person.

In one embodiment, the App can be used to track progress by asking selected questions from time to time, and asks questions when the person is distressed or is having triggering events. The app or software can add to and modify an existing BPS Formulation, and use biosensors to incorporate information such as heart rate, sweat, blood pressure, sleep, and activity tracking. In one embodiment, NLP (Natural Language Processing) can be used to incorporate existing medical records. All measures will be taken by the software to ensure patient health data confidentiality.

In one embodiment, the method for providing clinical support to user/patient's mental health by creating Bio-Psycho-Social (BPS) Formulation uses a computer-implemented system for providing the mental health assistance. The system comprises a computing device having at least one processor and a non-transitory memory unit coupled to the at least one processor configured to execute a set of instructions stored in the non-transitory memory unit, one or more databases in communication with the computing device via a communication network configured to store of psychiatric data history of users/patients, and a user device having an interactive user interface associated with the user/patient configured to deliver digital mental health service.

In one embodiment, the method provides BPS formulation, treatment, and tracking of mental health of the patient. In one embodiment, the method comprises the following steps. At one step, the system collects data that pertains to a patient's psychiatric history including data on the symptoms and medical, psychological, and social history in the least time-consuming way. At another step, the system collates the collected data into biological, psychological, and social categories using a database like structure. At another step, BPS is computed using algorithmic and/or Artificial Intelligence (AI). At another step, a unique phenotype or in colloquial language a fingerprint is created for an individual using BPS. At another step, the fingerprint is compared using the AI against a set of “stock” phenotypical representation of BPS associations with symptom complexes found in the patient that indicate types of patterns of BPS problems or associative chains. In one embodiment, the phenotypical representation is used to come up with the BPS formulation that is representative of the patient's problems and has solutions as per a matched phenotype. In one embodiment, the AI is configured to report and measure symptoms to reformulate BPS and provide feedback based on the reformulated BPS. At another step, a link between the symptoms that split into patients' symptom complex or diagnosis and BPS, and their improvements is analyzed. In one embodiment, the solution/suggestion is presented in the form of a Biopsychosocial plan which is a gold standard way to help the patient with psychiatric or psychological problem. In one embodiment, the system further tracks the patient's symptoms over time, and activates a solution/suggestion.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 shows a computer-implemented system executed in a network environment for treating and tracking mental health of a user/patient/customer using Bio-Psycho-Social (BPS) Formulation in an embodiment of the present invention.

FIG. 2 shows a biopsychosocial framework for providing holistic or 360° treatment in one embodiment of the present invention.

FIG. 3 shows a flowchart illustrating a process of a digital platform (i.e., PSYCHNOSIS application) for biopsychosocial analysis in one embodiment of the present invention.

FIG. 4 shows an example flowchart for performing biopsychosocial analysis for providing treatment suggestion in one embodiment of the present invention.

FIG. 5 shows a casual chain in one embodiment of the present invention.

FIG. 6 shows a flowchart illustrating a process of the measurement and treatment in a digital platform-based interventions in one embodiment of the present invention.

FIG. 7 shows a flowchart for reformulating BPS formulation in one embodiment of the present invention.

FIG. 8 shows a method for treating and tracking mental health of a user/patient/customer using Bio-Psycho-Social (BPS) Formulation in one embodiment of the present invention.

### DETAILED DESCRIPTION OF EMBODIMENTS

The present invention is best understood by reference to the detailed figures and description set forth herein.

It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

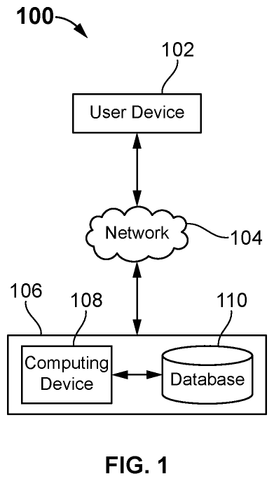

Referring to FIG. 1, a computer-implemented system executed in a network environment 100 for treating and tracking mental health of a user/patient/customer using Bio-Psycho-Social (BPS) Formulation, according to one embodiment of the present invention. The system runs in the computer-implemented environment 100 configured to provide an online/digital assistance for users suffering from mental health issues. In one embodiment, the system is a clinical support tool for users before they decide to go to family doctors, therapist, and psychiatrist. In one embodiment, the system is a digital platform designed to artificially replicate what a psychiatrist does when they are assessing a patient, in a user-friendly way.

In one embodiment, the network environment 100 comprises one or more user devices 102. The user device 102 has an interactive user interface or graphical user interface. Each user device 102 is associated with a user. The user may be an end-user, individual, patient, or customer. The customers may be general doctors, nurse practitioners (nurses who can prescribe) and social workers, therapists, psychologists, crisis workers, and internet therapists. In one embodiment, the user device 102 is installed with a digital platform (i.e., PSYCHNOSIS application). In one embodiment, PSYCHNOSIS is a type of e-mental health application. In one embodiment, the digital platform may be an application software or mobile application or web-based application or software application. The system further comprises a communication network 104 and a psychiatric data management system 106. The user device 102 is configured to deliver digital mental health services. In one embodiment, the user device 102 is enabled to access the psychiatric data management system 106 via the network 104. In one embodiment, the user device 102 enables the user to access one or more services provided by the system. In one embodiment, the user device 102 is at least any one of a smartphone, a mobile phone, a tablet, a laptop, a desktop, and/or other suitable hand-held electronic communication devices. In one embodiment, the user device 102 comprises a storage medium in communication with the network 104 to access the psychiatric data management system 106. In an embodiment, the network 104 could be Wi-Fi, WiMAX, wireless local area network (WLAN), satellite networks, cellular networks, private networks, and the like.

In one embodiment, the psychiatric data management system 106 comprises a computing device 108 and one or more databases 110 in communication with the computing device 108. In one embodiment, the computing device 108 is a server. In one embodiment, the computing device 108 could be a cloud server. In one embodiment, the server could be operated as a single computer. In some embodiments, the computer could be a touchscreen and/or non-touchscreen and adopted to run on any type of OS, such as iOS™, Windows™ Android™, Unix™, Linux™, and/or others. In one embodiment, the plurality of computers is in communication with each other, via networks. Such communication is established via any one of application software, a mobile application, a browser, an OS, and/or any combination thereof.

In one embodiment, the database 110 is in communication with the computing device 108 via the network 104. In one embodiment, the database 110 is accessible by the computing device 108. In another embodiment, the database 110 is integrated into the computing device 108 or separate from it. In some embodiments, the database 110 resides in a connected server or a cloud computing service. Regardless of location, the database 110 comprises a memory to store and organize certain data for use by the computing device 108.

In one embodiment, the computing device 108 comprises at least one processor and a non-transitory memory unit or computer-readable medium or memory unit coupled to the processor. The memory unit stores a set of instructions executable by the processor configured to analyze various clinical factors such as predispose, precipitate, perpetuate, and protect, and presumptive diagnosis. The memory unit could be RAM, ROM (including EPROM, EEPROM, PROM). In one embodiment, the user devices 102 are configured to access the services provided by the computing device 108 via the network 104. In one embodiment, the computing device 108 is configured to provide communication between the users and medical professionals in the digital platform to provide suggestions to the users to improve their mental health.

In one embodiment, the computing device 108 is configured to collect data that pertains to a patient's psychiatric history including data on the symptoms and medical, psychological, and social history in the least time-consuming way. The user interface is designed to emulate a human being or mental health professional asking such questions so as to have a pleasant and even therapeutic experience. Further, the system removes biases in questions.

In one embodiment, the computing device 108 is further configured to collate the collected data into biological, psychological, and social categories using a database like structure. In one embodiment, the computing device 108 is further configured to compute BPS using algorithmic and/or Artificial Intelligence (AI). BPS is the gold standard in psychiatric and psychological fields to holistically understand the person's current mental health issues such as to why this person is presenting with these problems at this point in time. This framework then is used to come up with a Bio-Psycho-Social Summary that would accurately reflect the user, for example, what the person is going through and what are the different factors that go into producing such a result. The central assertion is that there is value in understanding what is the issue affecting someone and the presentation of this is hypothesized to be therapeutic and extremely helpful in and of itself. In one embodiment, the BPS Formulation includes Statistical Manual of Mental Disorders (DSM) and International Classification of Diseases (ICD) and/or psychiatric diagnosis. In one embodiment, the BPS Formulation Summary is presented according to the end-user/consumer.

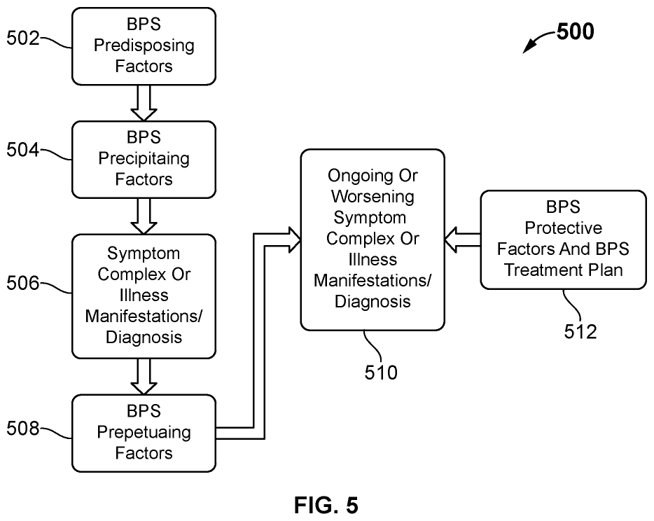

In one embodiment, the computing device 108 is further configured to create a unique phenotype or in colloquial language a fingerprint for an individual. This phenotypical representation is plotted in different dimensions to make a 2D or 3D picture representation. In one embodiment, the computing device 108 is further configured to compare the fingerprint using the AI or computer software against a database with known biopsychosocial associations. These “stock” phenotypical representation of BPS associations with symptom complex are commonly found in the Psychiatric literature that indicate types of patterns of BPS problems or associative chains. For example, Bio Predisposing factor of genetic depression history is linked with depression symptom complex and Alcohol precipitating or perpetuating for this symptom complex. This will then help to come up with a BPS formulation that is representative of the persons' problems and have solutions as per this “matched” phenotype. In one embodiment, a causal chain 500 (as shown in FIG. 5) for each linked factor between the symptom complex and BPS factor will use a rule-based database for performing pattern matching.

In one embodiment, the computing device 108 is further configured to analyze a link between the symptoms that split into patients' symptom complex or diagnosis with the BPS, and their improvements. In one embodiment, the computing device 108 is further configured to allow a medical professional to facilitate mental health treatment using the provided Biopsychosocial treatment plan based on the BPS Summary by the app, thereby improving patient's mental health immensely and preventing adverse outcomes. In one embodiment, the app is able to auto-populate the fields required in a medical professional's documentation, thereby saving time and the data can be exported to a preferred format for storing it, such as the Electronic Health Records of the patient in a particular healthcare system or hospital.

In one embodiment, the system is further configured to track the persons' symptoms over time and activates a solution/suggestion using the application software. The system further refines the unique fingerprint. Some factors of the unique fingerprint will not change over time and others will, either improving or getting worse. Each new problem will cause a spike in the fingerprint figure which can meet a threshold to activates a solution/suggestion by the application software. The solutions will be presented in the form of a Biopsychosocial treatment plan based on the BPS Formulation and Summary which is again the gold standard way to help someone with the psychiatric or psychological problem. The BPS plan will be configured based on the end user i.e., whether it is the patient or the health care provider. The figure is represented visually to give the user an idea of the patient's overall mental health. One can compare this to the “health indicator” on a video game and that to improve their health, the player must find a med kit which is the solution suggested by the App. Once this is implemented and the person is feeling better the fingerprint will change to show the progress to further help the patient or their health care provider with new suggestions needed to do to improve the patient's overall mental health. Then it is matched constantly again to the stock data to come up with new formulations and solutions. Also, there will be a stock fingerprint indicating normal mental health to compare against which is individual specific by analyzing what the realistic goals are for the particular person.

In one embodiment, the App can be used to track progress by asking selected questions from time to time, and asks questions when the person is distressed or is having triggering events. The app or software add and modify an existing BPS Formulation, and use biosensors to incorporate information such as heart rate, sweat, blood pressure, sleep, and activity tracking. In one embodiment, NLP (Natural Language Processing) can be used to incorporate existing medical records. All measures will be taken by the software to ensure patient health data confidentiality.

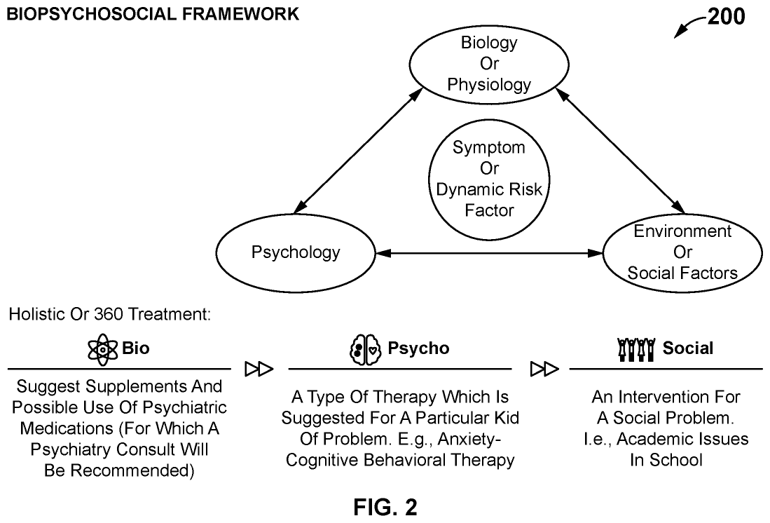

Referring to FIG. 2, a biopsychosocial framework 200 to provide holistic or 360° treatment, according to one embodiment of the present invention. The framework 200 presents solution for symptom or dynamic risk factor of biology or physiology, psychology, and environment or social factors. The solutions are presented in the form of a Biopsychosocial treatment plan, which is again the gold standard way to help someone with a psychiatric or psychological problem. For example, Bio—suggests supplements and possible use of psychiatric medications (for which a psychiatry consult will be recommended or if the end user is a doctor who can prescribe, suggestions for appropriate medications will be provided); Psycho—a particular type of psychotherapy which is suggested for that particular kind of problem, for example, anxiety—Cognitive Behavioral Therapy is suggested; Social—An intervention for that social problem, for example, for academic issues—accommodations in school.

On the B2B (Business to Business) feature, the app directs to a medical or psychological professional who may or may not be a part of a larger health care organization. The B2B can be activated whereby this App suggests other mental health Apps or physical providers through virtual or inpatient sessions. On the B2C (Business to Consumer) version, the app directs to the consumer or patient which will be a member of the general public who would like to learn why they are going through certain issues and what may be some ways to address these. For example, one can have a social media newsfeed, such as Facebook, with curated articles as per the BPS profile of the user and also have exercises and connections to mental health support resources and services and products. Optionally, the user may have social networking and connection to peers. For example, a Facebook like group can be formed with a group leader discussing a topic or providing therapy.

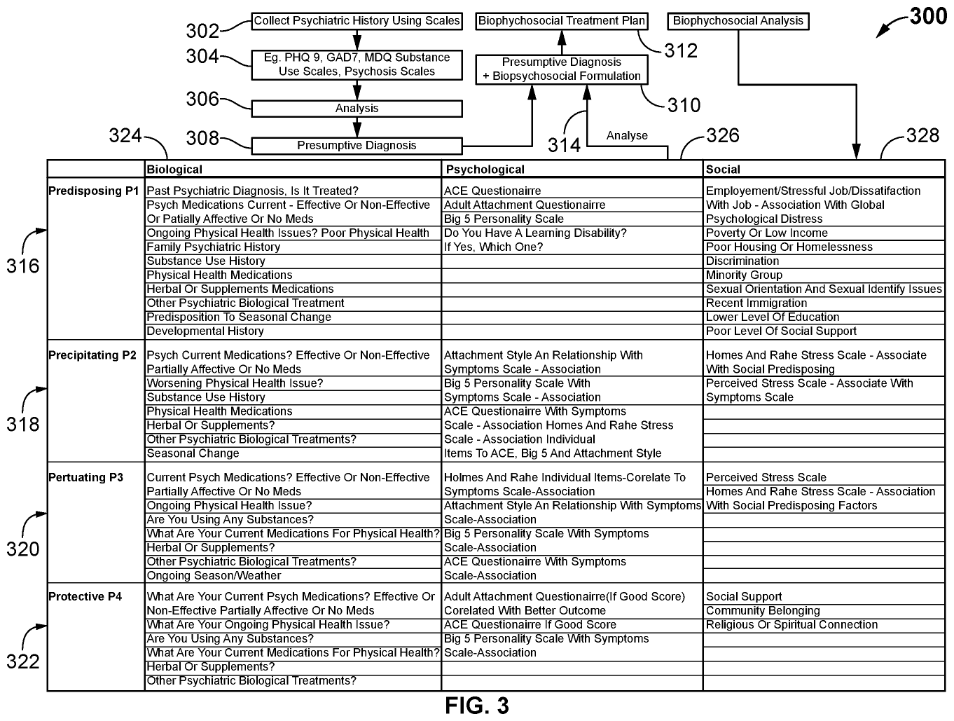

Referring to FIG. 3, a flowchart 300 illustrating a process of a digital platform (i.e., PSYCHNOSIS application) or app for biopsychosocial analysis, according to one embodiment of the present invention. The flowchart 300 comprises the following steps. At step 302, the app collects psychiatric history using different scales and questions pertaining to BPS factors. At step 304, the app uses standardized scales, for example, PHQ 9, GAD7, MDQ, substance use scales and psychosis scales, to collate a symptom complex(s) and compute presumptive diagnosis using the scores from these scales to give an estimate of the mental health symptoms which indicate a particular diagnosis, for example, clinical depression or major depressive disorder. At step 306, the app performs analysis to indicate presumptive diagnosis(es). At step 308, the app collects a set of presumptive diagnosis(es). In parallel to this process, Biological 324, psychological 326, and social 328 factors which are listed in the table are collected from the patient and categorized into a predisposing 316, precipitating 318, perpetuating 320, and protective 322 temporal and qualitative classification. At step 314, the app performs computer algorithm and/or AI based analysis to find associations between BPS Predisposing, Precipitating, Perpetuating and Protective factors for the patient and known associations in the psychiatric and psychological literature using a database structure which stores these known associations. Once an association is found, the interpretation of this association or its clinical relevance will be reported. At step 310, once a set of presumptive diagnosis are collected, the supporting information from the Biopsychosocial analysis 314, will be merged with it to create a BPS Formulation tied to the set of presumptive diagnosis(es). At step 312, the app formulates the biopsychosocial treatment plan. This process will be illustrated in the different examples below which show certain parts of the BPS analysis, an example of the complete analysis will then be shown by an illustrative example of a clinical case.

In an example, a patient is consuming 4 drinks of Alcohol per day. Association: Alcohol is a predisposing, precipitating and perpetuating factor for major depression. Therefore, interpretation is: Patient's depression is contributed to by ongoing use of Alcohol. Treatment suggestion is: Patient should cut down on alcohol consumption and patient should be referred to an Alcohol Addiction program (Psychological Treatment Plan). Also, alcohol anti-craving medications can be offered, for example, Naltrexone (Biological Treatment Plan).

In another example, Holmes and Rahe Scale questionnaire points towards a recent death of spouse. Association: Death of spouse is high stress event which is corelated with psychological predisposing factor of depression. Interpretation: Patients depression is contributed to by this factor. Treatment suggestion is to offer grief counselling psychotherapy to the patient.

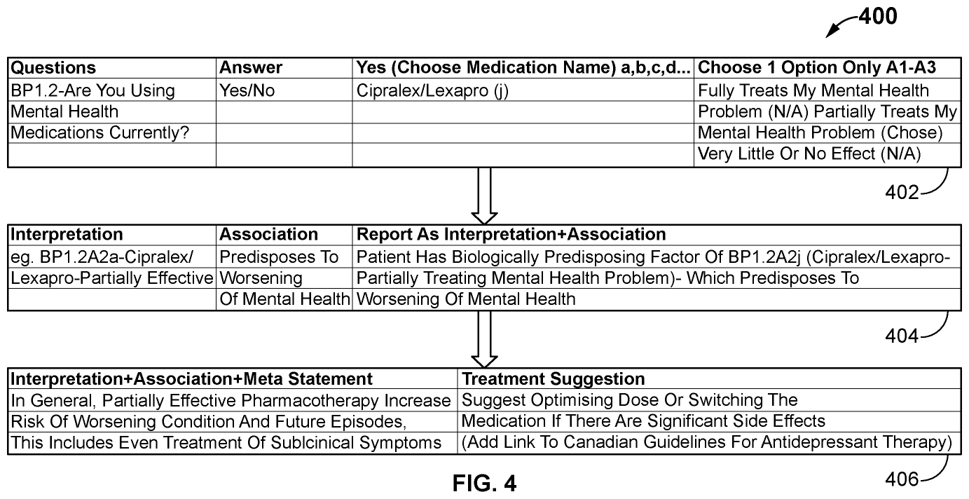

FIG. 4 shows an example flowchart 400 for performing a part of the biopsychosocial analysis for providing treatment suggestion confined to one biological factor of medication therapy, according to one embodiment of the present invention. At step 402, the app collects the medication history of the patient. For example, the patient is already on an antidepressant called Escitalopram or Lexapro. They are experiencing partial benefits from this antidepressant. At step 404, the association is determined, where the association is a predisposing factor for worsening depression as literature shows partially effective therapy is associated with this outcome. At step 406, the app presents a suggestion, where the Interpretation/Treatment Suggestion is to increase the dose.

The above information is used to create a unique fingerprint for the individual and the known associations present which could continue to affect the patient in this particular episode of illness and for future issues which may arise. Therefore, this fingerprint can be modified by repeating this process and future treatment and education can be implemented using the app. In the future, the patterns seen can be learned using a computer AI algorithm and real-time feedback and monitoring done to provide suggestions to both patient and clinician treating the patient.

In an example, a representative use case of PSYCHNOSIS mental health application is explained as follows. A patient, Mr. Lee (not an actual patient, only representative example) comes to general practitioner's (GP) office with a set of problems. Mr. Lee is asked to download the App which can gather data prior to his appointment to help his doctor understand his issues better and gather data for the BPS analyses and presumptive diagnosis. There is an option to do this at the GP office on a device, for example, an iPad, in a private room, or at home on patient's smartphone or computer. The App has interactive and pleasant multimedia interface using rich graphics. The App asks him to consent to gather some personal mental health information which will be stored in his Electronic Health Record (EHR) at his GP's and it is encrypted to ensure no data leakage. The App asks him questions regarding any safety concerns and this is not a concern currently. He picks an avatar of the virtual mental health assistant who will gather data.

Mr. Lee is greeted by a friendly voice of avatar Mr. Gray stating he is going to ask him some questions. There is also a prompt to include a family member to enter information. Questions focus on his set of problems as per the algorithm and the voice is empathetic providing support. There are different questionnaires on screen regarding mental health symptoms. Please note in an actual case there will be a lot more information found, this case has been simplified for illustration purposes. Standard clinical questionnaires are first presented such as PHQ9, GAD etc. A presumptive diagnosis of Major Depressive Disorder is computed. The App asks re Biopsychosocial factors contributing to his condition and the following are found:
Biologically—Low thyroid (Biological Precipitating and Perpetuating factor) and family history of depression in father (Biological Predisposing factor), and consumption of alcohol (Biological Precipitating and Perpetuating factor).
Psychologically—History of physical abuse and bullying as well as sexual abuse as per ACE Questionnaire (Psychological Predisposing factor).
Socially—Financial stress and bullying in the workplace and stress at home with family as per Holmes and Rahe Stress scale scores and subcategories. (Social Precipitating and Perpetuating factor). Good education with high socioeconomic status and spousal, family and friends support present (Social protective Factors).

At the office, GP sees a neatly typed report on Mr. XY's history which was gathered via weblink. Different subheadings regarding the patent's presenting problems and past history are listed in a format as used by health care practitioners (Chief Complaint, History of Presenting Illness, Past History, Family History, Past Personal History) and the analysis is given at the end of the report in the form of a Biopsychosocial Formulation and BPS Treatment plan.

### Biopsychical Formulation:

Mr. Lee is a 46 years Male who lives with his family and is a Bank Manager. In understanding his presenting problems of self-reported low mood and poor concentration, it is analyzed through the questionnaires that he meets clinical levels of Depression, suggestive of Major Depressive Disorder of moderate severity. He is also experiencing anxiety which meets clinical levels of Generalized Anxiety disorder and Alcohol use which may be problematic but not reaching levels of an Alcohol use Disorder at this time. He has a biological predisposition to depression due to the reported history of his father having significant depression. He has also suffered mild levels of depression in the past which is a further predisposing factor. He also has biologically predisposing and precipitating factors of hypothyroidism and mild alcohol misuse which is also probably perpetuating. His psychological predisposing factor is that he is a victim of childhood physical and mental abuse by bullying and sexual abuse. The psychologically precipitating and perpetuating factor can be workplace harassment he has indicated, which in turn can bring out the past trauma of bullying he sustained in childhood. His depression and anxiety seem to be temporally linked to his workplace stress which likely are worsened by the workplace stress he is reporting. He also has stress at home due to family issues. He also is enduring social stresses of workplace and financial stress which seem to be perpetuating factors. He has some protective factors of having a good education and high socioeconomic status and also good spousal and family support, and he has indicated he has supportive friends.

The following comprehensive Biopsychosocial plan is recommended:

Biologically—He will require blood tests to check for issues which can contribute to depression and different blood tests such as CBC for anemia, Vit B12 (which when low can interfere with cognition), Vit D, TSH for thyroid function and metabolic bloodwork. Please follow up on these results. It is likely indicated he will require antidepressants; SSRIs are the first line according to the Canadian and other International Guidelines. They are listed below with their common side effects and they have been cross-checked against his current prescription meds so they rule out the chance of interactions (which will be another feature in the app including list of medications suggested). There are also antidepressants and other medications which will help his anxiety which are presented in the report. He will need help with alcohol reduction or cessation to reduce this biological factor of his depression. The list of alcohol programs in the local community is listed in the report along with toll free numbers which can guide the patient to the right program. Also, Naltrexone as an Alcohol anti-craving can be considered for this patient. Optional genetic report on choosing the right medications and also understanding the genes of the patient that can predispose to mental illness and can also be accessed as part of advanced features in the app. A referral to a psychiatrist is recommended.

Psychologically—Cognitive Behavioral Therapy is indicated for this patient and listed in the report are local therapists, free and for a fee. Trauma therapy can also be considered.

Socially—Patient will need accommodations or time off from work as per your judgment (addressed to GP or health professional) to ease the stress he is experiencing. Financial counselling through a personal financial planner could also help. He may need workplace mediation through his HR program to resolve the issues at work. He is advised to bolster his social connections and take quality time with wife and kids and find support with child rearing at home.

The GP will benefit from a comprehensive safety assessment and a full report on history which is good medicolegal documentation stored in their EHR and for future reference for which they can share a link to the psychiatrist to facilitate documentation and time savings in hospital if the patient presents there. Mr. Lee is able to sign up for the app and have his report shared which has a non-medical plain language interpretation including pie charts that can monitor his symptoms and track his treatment and add data on a daily and as needed basis to reformulate Biopsychosocial Formulation and plan.

Mr. Lee uses the real time monitoring and friendly feedback to bolster his current treatment. He gets information on current antidepressant and articles educating him on stress management and alcohol use. He is working on the recommended BPS plan tailored specifically to him as a patient and connects with the therapist for CBT and an alcohol counsellor. He is working with HR in the company and also trying to make changes at home with help of his wife. Once a psychiatrist appointment occurs in 2 months, the gathered information from the app helps the interview process. The patient gets more time with the psychiatrist explaining diagnosis and treatments and the psychiatrist has to document a lot less. This can enable them to see more patients and have an easier patient interview process.

Referring to FIG. 5, a casual chain 500, according to one embodiment of the present invention. The casual chain 500 comprises one or more clinical factors including, but not limited to, BPS predisposing factors 502, BPS precipitating factors 504, symptom complex or illness manifestations/diagnosis 506, and BPS perpetuating factors 508. These factors are used for ongoing or worsening symptom complex or illness manifestations/diagnosis 510. Further, the casual chain 500 has BPS protective factors and BPS treatment plan 512. In one embodiment, the casual chain 500 focuses on symptoms and their treatment without understanding the BPS formulation which is the gold standard.

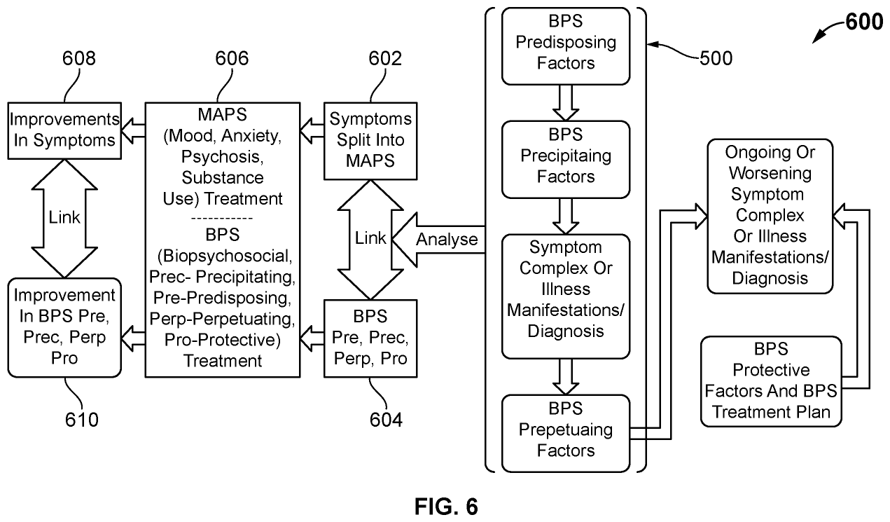

Referring to FIG. 6, a flowchart 600 illustrates a process of the measurement and treatment in a digital platform-based interventions, according to one embodiment of the present invention. The flowchart 600 establishes the casual chain 500 for each linked factor between symptom complex and BPS factor. It uses a rule-based database which will do pattern matching using an algorithm. In one embodiment, the algorithm may be heuristic or pattern matching. The clinical factors in the casual chain 500 are used to analyze the link between the symptoms that split into MAPS 602 (symptoms of mood, anxiety, psychosis, and substance use) and BPS clinical factors (predisposing, precipitating, perpetuating, and protecting) 604 that are used for MAPS treatment and BPS treatment plan 606 respectively. The MAPS treatment and BPS treatment 606 are fed to the improvement in systems 608 and improvement in BPS clinical factors (predisposing, precipitating, perpetuating, and protecting) 610 that are linked together.

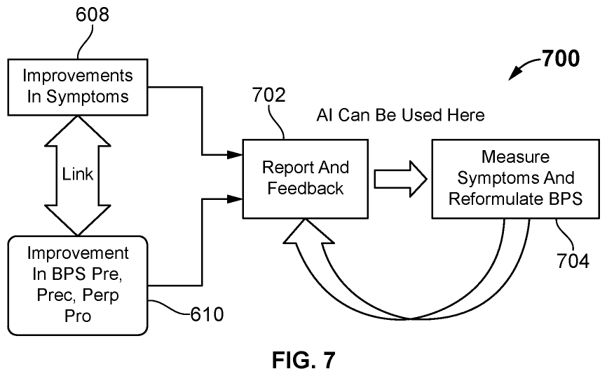

Referring to FIG. 7, a flowchart 700 for reformulating BPS formulation, according to one embodiment of the present invention. The improvement in systems 608 and improvement in BPS clinical factors (predisposing, precipitating, perpetuating, and protecting) 610 that are linked together uses the AI algorithm to measure symptoms and reformulates BPS 704. The AI provides report and feedback 702 based on the measured symptoms and reformulates BPS 704.

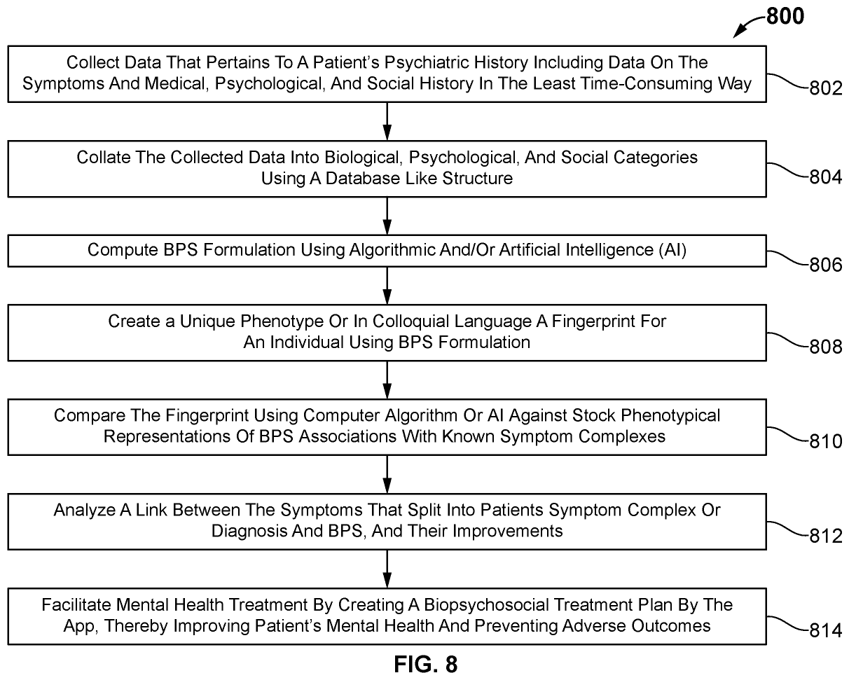

Referring to FIG. 8, a method 800 for providing clinical support to user/patient's mental health by creating Bio-Psycho-Social (BPS) Formulation, according to one embodiment of the present invention. The method 800 uses a computer-implemented system for providing mental health assistance. The system comprises a computing device having at least one processor and a non-transitory memory unit coupled to the at least one processor configured to execute a set of instructions stored in the non-transitory memory unit, one or more databases in communication with the computing device via a communication network configured to store of psychiatric data history of users/patients, and a user device having an interactive user interface associated with the user/patient configured to deliver digital mental health service.

In one embodiment, the method 800 provides BPS formulation, treatment, and tracking of mental health of the patient. In one embodiment, the method 800 comprises the following steps. At step 802, the system collects data that pertains to a patient's psychiatric history including data on the symptoms and medical, psychological, and social history in the least time-consuming way. At step 804, the system collates the collected data into biological, psychological, and social categories using a database like structure. At step 806, BPS Formulation is computed using algorithmic and/or Artificial Intelligence (AI) or computer algorithm. At step 808, a unique phenotype or in colloquial language a fingerprint is created for an individual using BPS. At step 810, the fingerprint is compared using the AI against a set of “stock” phenotypical representation of BPS associations with symptom complex that indicate types of patterns of BPS problems or associative chains in the psychiatric literature. In one embodiment, the phenotypical representation is used to come up with the BPS formulation that is representative of the patient's problems and has solutions as found in a database which has solutions listed for each type of mental health issue as known in the psychiatric literature. In one embodiment, the AI is configured to report and measure symptoms to reformulate BPS Formulation and provide feedback based on the reformulated BPS Formulation. At step 812, a link between the symptoms that split into patients' symptom complex or diagnosis and BPS Factors, and their improvements is analyzed. At step 814, the system creates a BPS Treatment plan based on the BPS Formulation. In one embodiment, the system allows the medical professionals to facilitate a BPS treatment plan, thereby improving patient's mental health immensely and preventing adverse outcomes. In one embodiment, the solution/suggestion is presented in the form of a Biopsychosocial plan which is a gold standard way to help the patient with psychiatric or psychological problem. In one embodiment, it informs and educates the patient regarding their unique BPS Factors and BPS Formulation. In one embodiment, the system further tracks the patient's symptoms over time, and activates a solution/suggestion.

The applications of the present invention include, but not limited to the following: The primary application is to assist General Practitioners, Family doctors or Nurse Practitioners in obtaining a Biopsychosocial Plan for their patients prior to them seeing a psychiatrist. They are not trained in this BPS format and provide usually fragmented care. They rely heavily on input from a psychiatrist before initiating or improving mental health care. In most countries, the wait times for accessing a psychiatrist can be 1-6 months or more. During this time the patient does not receive timely care which is based on BPS plan which is the gold standard plan a psychiatrist would initiate. Having the benefit of this analysis from this clinical assist tool which is computer algorithm and AI based can help the patient begin their treatment journey which would improve outcomes immensely and prevent adverse outcomes such as worsening of illness, suicide, legal issues, family and caregiver burnout, workplace absenteeism and dissatisfaction with the medical system. The user interface is designed to emulate a human being or mental health professional asking such questions to have a pleasant and even therapeutic experience, and removes biases in questions. The app can be used to break the formulation gently to people, in a non-judgmental way without medical jargon. A human Psychiatrist's background can often lead to subconscious biases which are harmful, and the app can try to eliminate this issue. Some people are afraid to approach a mental health professional and this can be a potential solution. This would be helpful to service people who do not have access to psychiatrists in their local area, all over the world as mental health help can be hard to find or is expensive. This can be used as a research information gathering application. Professional edition will check drug interactions and recommend blood tests. It can recommend social and psychological services in the area for easy access. The latter functionality would enable the App to partner with existing businesses which connect people to therapists and mental health services. This app can help organizations or practitioners save time on history gathering, auto populate electronic health records and also provided a BPS summary with a BPS plan which is according to the findings of the BPS formulation (which is again the normal procedure for a human mental health professional). Further, this app can help organizations or practitioners save time on history gathering, auto populate electronic health records and provided a BPS summary with a BPS plan which is according to the findings of the BPS formulation.

The advantages of the present invention include, but not limited to the following: The system of the present invention uses a computer software for collecting information, reviewing it and then storing it. It increases data accuracy and increases time spent on value-add services by the specialist/doctor like therapy or psychoeducation and providing support or explanation of medication related issues or psychotherapy advised. The app will have multi-language facility. The patient may provide answers to the questions at the speed based on patient desire, at the time of the day when they have free time, in the language they find easiest, in the space they are most comfortable in. It is easy to involve family and friends who may have to provide input using their phone, where family can add additional or collateral information. It is for globally good and adds value for doctors, patients and the government (the payer). In addition, long form of voice and text input is used to gather additional data after patient has started using the UI which can make empathetic statements and uses transitions, which are gentle changes when moving from one line of questioning to another or warnings about asking certain sensitive information such as history of abuse. The app could also be used as an initial basic questionnaire tool to save time for emergency/crisis workers and psychiatrists, inpatient psychiatrists, community psychiatrists or nurses or family doctors or social workers. The app allows the patient to sign on the consent form for privacy, which includes Privacy law compliance. The system also develops a community of doctors, psychologists, and medical workers and users. Further, the data can be saved on the local device, powered by a copy of the cloud software or blockchain technology.

Preferred embodiments of this invention are described herein, including the best mode known to the inventors for carrying out the invention. It should be understood that the illustrated embodiments are exemplary only and should not be taken as limiting the scope of the invention.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A computer-implemented system for providing clinical support to user/patient's mental health by creating Bio-Psycho-Social (BPS) Formulation, comprising:
a computing device having at least one processor and a non-transitory memory unit coupled to the at least one processor, wherein the processor is configured to execute a set of instructions stored in the non-transitory memory unit;
one or more databases in communication with the computing device via a communication network configured to store of psychiatric data history of users/patients, and
a user device having an interactive user interface associated with the user/patient configured to deliver digital mental health service, wherein the computing device is configured to,
collect data that pertains to a patient's psychiatric history including data on the symptoms and medical, psychological, and social history in the least time-consuming way;
collate the collected data into biological, psychological, and social categories using a database like structure;
compute BPS using algorithmic and/or Artificial Intelligence (AI);
create a unique phenotype or in colloquial language a fingerprint for an individual user's BPS;
compare the fingerprint using the AI against a set of “stock” phenotypical representations of BPS associations with the symptom complex found in the patient that indicate types of patterns of BPS problems or associative chains;
analyze a link between the symptoms that split into patients' symptom complex or diagnosis and BPS, and their improvements, and
allow a medical professional to facilitate a BPS treatment plan, thereby improving patient's mental health immensely and preventing adverse outcomes.

2. The system of claim 1, is configured to provide BPS formulation, treatment, and tracking of mental health of the patient.

3. The system of claim 1, wherein the user device is configured to communicate with the computing device via the communication network using an application software or mobile application or web-based application, or desktop application executed in a computer-implemented environment or network environment.

4. The system of claim 1, wherein the BPS formulation is used to present a BPS formulation summary according to end-users/customers/patients, which accurately reflect the user using different factors.

5. The system of claim 1, wherein the phenotypical representation is used to come up with the BPS formulation that is representative of the patient's problems and have solutions as per a matched phenotype.

6. The system of claim 1, wherein the AI is configured to report and measure symptoms to reformulate BPS and provide feedback based on the reformulated BPS.

7. The system of claim 1, further tracks the patient's symptoms over time, and activates a solution/suggestion.

8. The system of claim 7, wherein the solution/suggestion is presented in the form of a Biopsychosocial plan which is a gold standard way to help the patient with a psychiatric or psychological problem.

9. The system of claim 1, further tracks progress of mental health by asking selected questions from time to time, and asks questions when the user is distressed or having triggering events.

10. The system of claim 1, further adds and modifies an existing BPS Formulation, and use biosensors to incorporate information such as heart rate, sweat, blood pressure, sleep, and activity tracking.

11. The system of claim 1, wherein the patient's psychiatric data are stored in a local device or using a patient privacy and confidentiality maintaining technology.

12. The system of claim 1, helps organizations or practitioners to gather patient's psychiatric history in less time, auto populate electronic health records, and provide a BPS summary with a BPS plan according to the findings of the BPS formulation.

13. A method for providing clinical support to user/patient's mental health by creating Bio-Psycho-Social Formulation (BPS) using a computer-implemented system comprises a computing device having at least one processor and a non-transitory memory unit coupled to the at least one processor configured to execute a set of instructions stored in the non-transitory memory unit, one or more databases in communication with the computing device via a communication network configured to store of psychiatric data history of users/patients, and a user device having an interactive user interface associated with the user/patient configured to deliver digital mental health service, wherein the method comprises the steps of:
collecting data that pertains to a patient's psychiatric history including data on the symptoms and medical, psychological, and social history in the least time-consuming way;
collating the collected data into biological, psychological, and social categories using a database like structure;
computing BPS using algorithmic and/or Artificial Intelligence (AI);
creating a unique phenotype or in colloquial language a fingerprint for an individual using the BPS formulation;
comparing the fingerprint using AI or a computer algorithm against a set of “stock” phenotypical representations of BPS associations with symptom complex, found in the patient that indicate types of patterns of BPS factors interacting with common psychiatric/mental health symptoms which are also called associative chains;
analyzing a link between the symptoms that split into patients' symptom complex or diagnosis and BPS factors, and their improvements, and
allowing a medical professional to review a BPS Formulation and based on this, facilitate a BPS treatment plan, thereby improving patient's mental health immensely and preventing adverse outcomes.

14. The method of claim 13, is performed to provide BPS formulation, treatment, and tracking of mental health of the patient.

15. The method of claim 13, wherein the phenotypical representation is used to come up with the BPS formulation that is representative of the patient's problems and these have solutions as per a matched phenotype which is already present in a database.

16. The method of claim 13, wherein the AI or computer algorithm is configured to report and measure symptoms to reformulate BPS and provide feedback based on the reformulated BPS Formulation.

17. The method of claim 13, further tracks the patient's symptoms over time, and activates a solution/suggestion.

18. The method of claim 17, wherein the solution/suggestion is presented in the form of a new Biopsychosocial plan which is a way to help the patient with psychiatric or psychological problem on an ongoing basis.

19. The method of claim 13, further tracks progress of mental health by asking selected questions from time to time, and asks questions when the user is distressed or having triggering events.

20. The method of claim 13, further adds and modifies an existing BPS Formulation, and use biosensors to incorporate information such as heart rate, sweat, blood pressure, sleep, and activity tracking.
